(19) United States
(12) Patent Application Publication
(10) Pub. No.: US 2015/0005387 A1
(43) Pub. Date: Jan. 1, 2015

(54) COMPOSITION CONTAINING AGMATINE, AND USES THEREOF IN PREPARING DRUGS OR NEUTRACEUTICAL SUBSTANCES

(71) Applicant: Jacques-Philippe Moulinoux, Rennes (FR)

(72) Inventor: Jacques-Philippe Moulinoux, Rennes (FR)

(73) Assignee: NUTRIALYS MEDICAL NUTRITION SA, St Gregoire (FR)

(21) Appl. No.: 14/371,529

(22) PCT Filed: Jan. 10, 2013
(86) PCT No.: PCT/FR2013/050061

§ 371 Date: Aug. 11, 2014

(30) Foreign Application Priority Data
Jan. 10, 2012 (FR) 12/50259

(51) Int. Cl. A61K 31/155; A61K 45/06; A23L 1/305; A61K 31/132

(52) U.S. Cl. CPC A61K 31/155; A61K 31/132; A61K 45/06; A23L 1/3051; A23V 2002/00
USPC 514/634

(57) ABSTRACT

The present invention relates to novel compositions including a mixture of cadaverine, putrescine, spermine and spermidine at a concentration ranging from 0.31 to 0.63 nmol per gram of composition, the composition also including agmatine at a concentration ranging from 120 to 17100 nmol per gram of composition, and uses thereof in the treatment, in a patient, of pathologies associated with cellular hyperproliferation.

The present invention relates to novel compositions containing agmatine, and uses thereof in the preparation of medicaments or nutraceuticals.

In prokaryotes and eukaryotes, the polyamines are organic molecules composed solely of carbon, nitrogen and hydrogen atoms and containing at least two amine functions. These polycationic aliphatic amines include putrescine, spermidine and spermine in eukaryotes. Cadaverine, another polyamine, is only present in prokaryotes.

The polyamines are synthesized from L-arginine or L-methionine. Their biosynthesis and catabolism pathway is based on a limited number of enzymes. The synthesis of putrescine starting from ornithine (degradation product of arginine) involves Ornithine DeCarboxylase (ODC). The second synthesis pathway of putrescine starting from agmatine involves agmatinase. Spermidine synthase (SpdS) allows the synthesis of spermidine starting from putrescine, and spermidine will in its turn be used by spermine synthase (SpmS) to form spermine Spermidine/spermine N1-acetyltransferase (SSAT) constitutes the key enzyme in the catabolism of the polyamines which allows retroconversion of spermine to spermidine and spermidine to putrescine. SSAT allows fine regulation of the intracellular levels of the various polyamines that may prove toxic to the cell. Spermidine and spermine can also be synthesized from methionine by the action of S-adenosylmethionine decarboxylase (SAMdc).

Data in the literature show that a daily food ration of 2000 kcal (kilocalories) supplies a dose of biologically “active” polyamines that ranges from 200 000 to 700 000 nanomoles (nmol). This means that a daily food ration supplies an intake of 100 to 350 nmol of biologically active polyamines per kilocalorie (Bardocz et al., 1995, Br. J. Nutr., 73(6): 819-828). This difference depends on the nature of the foodstuffs consumed, which contain variable quantities of polyamines.

To be able to convert quantities of biologically active polyamines expressed in nanomoles into grams (g), it is necessary to consider an average molecular weight for all of the biologically active polyamines. This average molecular weight of the biologically active polyamines is an approximation and is estimated at 145.24 g/mol.

Thus, a daily ration of 2000 kcal supplies from 14.5 to 51 μg of biologically active polyamines/kcal. This corresponds to an intake of 29 to 102 mg of biologically active polyamines/day or to an intake of 29 to 102 mg of biologically active polyamines per 2000 kcal.

It may prove necessary to consider the level of each of the polyamines considered separately, rather than the pool of biologically active polyamines taken together. Foodstuffs can be divided into three classes according to their polyamines content (www.guerir.org/magazine/guide-nutritionnel-nutrialys.pdf). This classification makes it possible to estimate the approximate levels of each of the polyamines (cadaverine, putrescine, spermidine and spermine) for each category of foodstuffs. Thus, an “average” daily food ration supplies about 10.8 mg of putrescine, about 4.4 mg of cadaverine, about 15.7 mg of spermidine and about 6.7 mg of spermine, respectively, per 2000 kcal. This corresponds to an overall level of polyamines (cadaverine, putrescine, spermidine and spermine) of about 37.6 mg for an “average” daily food ration of 2000 kcal. Consequently, putrescine represents 28.7%, cadaverine represents 11.7%, spermidine represents 41.8%, and spermine represents 17.8% of the overall level of polyamines in the aforesaid food ration.

The biologically “active” polyamines, or their analogues (organic chemical substances structurally similar to the polyamines) or biologically “active” derivatives thereof (chemical substances related structurally to a polyamine, and that theoretically are obtainable from the latter) can be identified in particular by at least one of the following methods:

1) In Culture:

A biologically “active” polyamine or a biologically “active” analogue or derivative thereof must be able to participate in the physiological cellular polyamine metabolism, or be capable of interfering with the latter or else of causing dysregulation thereof
a. A biologically “active” polyamine or a biologically “active” analogue or derivative thereof must therefore be able to bind to or be recognized by the transport system or systems that aim to internalize it in a living cell. Addition of a radiolabelled biologically “active” polyamine or of a radiolabelled biologically “active” analogue or derivative thereof to the culture medium allows its internalization to be verified.
b. A biologically “active” polyamine or a biologically “active” analogue or derivative thereof must be able to suppress the inhibition of cellular proliferation caused by inhibition of the endogenous anabolism of the polyamines (e.g. by α-DFMO, alpha-difluoromethylornithine).
c. A biologically “active” polyamine, or a biologically “active” analogue or derivative thereof, including synthetic, causing dysregulation of the natural polyamine metabolism, must be able to modulate the level of cellular proliferation.

2) In Vivo:

In an animal with a grafted tumour, the exogenous intake (gastrointestinal tract) of biologically “active” polyamines must suppress the beneficial anticancer effects caused by the deficiency of biologically “active” polyamines induced by the decrease in endogenous and exogenous sources of biologically “active” polyamines, wherein this exogenous intake may or may not be coupled with anticancer medicaments.

Thus, in the eukaryotes, putrescine, spermidine and spermine have the properties of the biologically active polyamines as defined above and are therefore considered as such in the present invention.

The polyamines are compounds that are essential for growth and cellular differentiation. The inventors have shown that a diet depleted of polyamines was able to potentiate the effects of irreversible inhibitors of ODC on tumour growth (EP 0 703 731 B1).

Moreover, studies have shown that cancer cells contain higher levels of polyamines than those observed in healthy cells (WO 1997/11691). Depletion of polyamines has been used in anticancer strategies. This depletion is obtained either by inducing expression of SSAT or stabilization of its messenger RNA by an analogue of spermine, in particular by N¹N¹²-diethylnorspermine (DENSPM) (Parry L et al., 1995, Biochem. J. 305: 451-458), or by inhibiting ODC using reversible or irreversible inhibitors such as α-DiFluoroMethylOrnithine (α-DFMO) (WO 2002/15895), or by blocking polyamine transport at the cell membrane level.

Although no particular gene has been identified as the polyamine transporter, three distinct polyamine transport systems have been demonstrated in mammals (Igarashi et al., 2010, Plant Physiology and Biochemistry, 48, 506-512). Antizyme, a protein involved in the degradation of ODC, is able to modulate entry or exit of polyamines into and out of the cell by a mechanism that has yet to be elucidated. A mechanism of endocytosis of the polyamines mediated by glypican-1 and caveolin-1 has also been reported. More recently, studies have demonstrated the presence of a protein complex consisting of the proteins SLC3A2 and y⁺LAT capable of excreting putrescine from the cell and of causing arginine to enter in exchange.

The development of inhibitors of polyamine transport forms the subject of many investigations. Various classes of molecules have been developed, in particular spermine analogues (Burns M. R., 2009, J. Med. Chem., 52, 1983-1993) or polyamine dimers (US 2005/0267220 A1), optionally bound to an anthracene nucleus (WO 2010/148390).

The inhibitors of the anabolic enzymes of the polyamines and the inhibitors of polyamine transport are essentially involved in the endogenous synthesis of the polyamines by the body's cells.

The bacteria of the intestinal flora constitute the second source of endogenous synthesis of the polyamines. The use of antibiotics targeting these bacteria can reduce this other endogenous source of polyamines.

Agmatine, resulting from the decarboxylation of arginine by arginine decarboxylase (ADC), is a precursor of putrescine. Agmatine homeostasis is maintained by the liver. Investigation of the tissue distribution of agmatine in the rat shows that agmatine is present, at variable levels, in most of the body's tissues. The highest levels of agmatine are found in the stomach, the intestines, the aorta and the spleen. Agmatine is also found, at a lower concentration, in other tissue compartments and in particular the liver, the lungs, the kidneys and the brain (Raasch et al., 1995, Ann. N.Y. Acad. Sci. July 12; 763: 330-4). The presence of agmatine throughout the tissues means it has various physiological functions.

Agmatine is considered to be a neurotransmitter or a neuromodulator in mammals. Agmatine is able to bind to various types of receptors, in particular to the imidazoline receptors, which are involved in the control of blood pressure. Thus, intravenous injection of agmatine at doses from 1 to 100 mg/kg leads to a decrease in blood pressure by inhibiting noradrenaline release (Raasch et al., 2002, British Journal of Pharmacology 135, 663-672).

Agmatine is also an antagonist of the N-methyl-D-aspartate (NMDA) and α2-adrenergic receptors, which are involved in the perception of chronic pain (Regunathan S, 2006, A.A.P.S Journal, 8(3): E479-E484). Agmatine has an anti-allodynic effect that allows animals injected with 0.3 nmol of agmatine to regain normal perception of pain (EP 1 014 959 B1). Moreover, the Courteix team showed that an intrathecal injection of agmatine suppresses tactile or thermal allodynia as well as hyperalgesia in diabetic rats (Courteix et al., 2007, J. Pharmacol. Exp. Ther., 322(3): 1237-1245). More recently, a clinical study conducted on patients with a herniated disc shows that taking 2.67 g/day of agmatine sulphate for 14 days causes attenuated perception of pain in these patients (WO 2010/030470).

Agmatine is in addition involved in the proliferation of cancer cells. Satriano's team showed that by dose-dependent inhibition of ODC, agmatine blocks the synthesis of polyamines in the renal cells, thus preventing their proliferation (WO 1998/13037).

Moreover, samples taken from tissues originating from colon carcinoma showed a level of agmatine below that of the adjacent healthy tissues (Molderings et al., 2004, Cancer, 101: 858-868). This is a direct consequence of inhibition of expression of ADC in the neoplastic tissues (Haenisch et al., 2008, Am. J. Physiol. Gastrointest. Liver Physiol., 295: G1104-G1110).

Agmatine also acts directly on the cell cycle. In vitro treatment of cell lines from hepatocarcinomas with doses of agmatine ranging from 10 μm to 1 mM leads to stopping of the cancer cells in phase G2/M of the cell cycle, but without leading to their apoptosis (Gardini et al., 2003, Journal of Hepathology, 39: 793-799). This cytoprotective effect of agmatine was confirmed on cellular models of colon cancer (Mayeur et al., 2005, B.B.A., 745: 111-123) and in retinal ganglion cells where apoptosis had been induced by TNF-α or by hypoxia (US 2010/0130783).

Finally, a specific agmatine transport system has been demonstrated at mitochondrial level, suggesting a regulatory effect of agmatine on mitochondrial function (Alvi et al., 2006, Biochem. J., 396: 337-345; Agostinelli et al., 2010, Amino Acids, 38: 393-403).

In the context of cancer treatment, increase in patient survival or length of life is still a major preoccupation. There is consequently a real need to develop new compositions that can contribute to the treatment of pathologies associated with cellular hyperproliferation, and in particular to cancer treatment.

Surprisingly, the inventors demonstrated a beneficial effect of agmatine on the metabolism of cancer cells, which makes it possible to distinguish it from biologically active polyamines participating in cellular homeostasis.

One of the aims of the invention is to supply compositions for use in the treatment of pathologies associated with cellular hyperproliferation, in particular in the treatment of cancer.

Another aim of the invention is to supply new compositions containing agmatine that are more effective than the compositions described in the prior art in the context of the treatment of pathologies associated with cellular hyperproliferation, in particular in the treatment of cancer.

Another aim of the invention is to propose compositions that can be administered to patients in combination with (chemotherapeutic) anticancer treatment leading to a synergistic effect in terms of cancer treatment.

The present invention relates to compositions comprising a mixture of cadaverine, putrescine, spermine and spermidine at a concentration ranging from 0.31 to 0.63 nmol per gram of composition, said composition also comprising agmatine at a concentration ranging from 120 to 17100 nmol per gram of composition.

Thus, the concentration of agmatine is greater than that of the biologically active polyamines in the compositions of the present invention. Moreover, the overall level of the biologically active polyamines in the compositions of the present invention is far lower than that present in the foodstuffs making up an “average” food ration.

Surprisingly, the inventors have shown for the first time in vivo the existence of a synergistic effect in the treatment of pathologies associated with cellular hyperproliferation, based on the combination of compositions comprising on the one hand a mixture depleted of cadaverine, putrescine, spermidine and spermine and on the other hand agmatine at a concentration higher than that supplied by an average food ration composed only of foodstuffs. “Foodstuff” means any substance or product, whether processed, partially processed or unprocessed, intended to be ingested by humans (EC Regulation No. 178/2002, Article 2). Foodstuffs are classified conventionally in six groups, comprising: milk and milk products, fish and meat, fats, cereals and derivatives, fruit and vegetables, and carbohydrates and their derivatives.

“Agmatine” denotes the precursor of putrescine corresponding to the formula NH₂—(CH₂)₄—N═C(—NH₂)₂. An “average” daily food ration of 2000 kcal consisting exclusively of foodstuffs supplies about 1.3 mg of agmatine. Agmatine not having any of the characteristics permitting identification of a polyamine that is biologically active in vitro is not considered as such in the present invention.

“Mixture of cadaverine, putrescine, spermidine and spermine” denotes the combination of the four natural polyamines corresponding respectively to the formulae NH₂—(CH₂)₅—NH₂, NH₂—(CH₂)₄—NH₂, NH₂—(CH₂)₃—NH—(CH₂)₄—NH₂ and NH₂—(CH₂)₃—NH—(CH₂)₄—NH—(CH₂)₃—NH₂ in a composition potentially containing other excipients.

The concentrations of the various polyamines are expressed in nanomoles (nmol) per gram of composition. The terms nanomoles and nmol are equivalent and may be used indiscriminately hereinafter. As an example, this means that in a gram of composition the concentration of agmatine may range from 120 to 17100 nanomoles.

The compositions of the present invention can be ingested by a mammal “Mammal” denotes a human being or an animal, wherein said animal may be a pet animal or a farm animal.

The compositions of the invention display an efficacy that may depend on the stage of anticancer treatment being followed by the patient.

This efficacy may be optimum when the compositions are used as the sole exogenous source of biologically active polyamines (putrescine, spermine and spermidine) and of agmatine. In this case, control of the concentrations of these various elements is total. In fact, the patient's diet contains a level of biologically active polyamines that is as low as possible. In parallel, the patient receives, by means of the compositions of the invention, a food ration highly enriched with agmatine.

When the compositions of the present invention are combined with foodstuffs, control of the concentrations of biologically active polyamines becomes trickier. Thus, so as not to counteract the effects of the compositions of the present invention, it seems necessary for the foodstuffs to be selected on the basis of their biologically active polyamines content. Consequently, the foodstuffs selected must contain the lowest possible levels of biologically active polyamines. The aim is that the level of biologically active polyamines supplied by the compositions of the invention is altered the least by the introduction of foodstuffs in the patient's diet.

Conversely, as the intake of agmatine in the compositions of the invention is greater than that of an average food ration composed solely of foodstuffs, the agmatine contained in the foodstuffs will have little effect on the overall concentration of agmatine in the patient's diet consisting of a mixture of compositions of the invention and foodstuffs.

According to a particular embodiment, the concentration of agmatine in the compositions of the invention ranges from 120 to 1450 nmol per gram of composition.

According to a more particular embodiment, the concentration of agmatine in the compositions of the invention ranges from 1450 to 3000 nmol per gram of composition.

According to an even more particular embodiment, the concentration of agmatine in the compositions of the invention ranges from 3000 to 6000 nmol per gram of composition.

According to another embodiment, the concentration of agmatine in the compositions of the invention ranges from 6000 to 17100 nmol per gram of composition.

Advantageously, the concentration of putrescine in the compositions of the invention ranges from 0.19 to 0.32 nmol per gram of composition.

More advantageously, the concentration of cadaverine in the compositions of the invention ranges from 0.02 to 0.08 nmol per gram of composition.

More advantageously, the concentration of spermine in the compositions of the invention ranges from 0.009 to 0.03 nmol per gram of composition.

Even more advantageously, the concentration of spermidine in the compositions of the invention ranges from 0.09 to 0.20 nmol per gram of composition.

When the compositions of the invention constitute the patients' sole or principal food source, these compositions may be enriched with fats, proteins, carbohydrates, vitamins, minerals and electrolytes in quantities such that the patient does not suffer from malnutrition or deficiencies.

The compositions of the invention contain, in percentage dry weight relative to total dry weight: 10% to 35% fats, 8% to 30% proteins, 35% to 80% carbohydrates, and up to 10% of a mixture consisting of vitamins, minerals and electrolytes.

“Food source” denotes all forms of diet, i.e. all of the foodstuffs that may constitute the nutrition of a human or of an animal, a diet composed of meal substitutes, or any other source of food allowing a human or animal to be kept alive.

“Mixture consisting of vitamins, minerals and electrolytes” denotes the vitamins and minerals performing a defined role in the body. The vitamins may be selected from a group consisting of vitamin A, vitamin B1, B6, B12, vitamin C, vitamin D3, vitamin K1, riboflavin, pantothenic acid, niacin, folic acid, biotin, choline, inositol. The minerals and electrolytes may be selected from a group consisting of sodium, potassium, calcium, phosphorus, magnesium, iron, zinc, copper, manganese, chlorides, iodine, selenium, chromium, molybdenum. The choice of the vitamins, minerals and electrolytes is not to be restricted by the lists given above. A person skilled in the art can adapt the proportions of each of these constituents, allowing the patient to receive balanced nutrition, which meets his daily nutritional requirements.

The polyamines in the body are derived from three main sources: cellular proliferation (physiological and tumoral), nutrition, and the intestinal bacteria. For maximum control of the intake of polyamines to the body, it may prove necessary to limit not only the exogenous intake by means of perfectly controlled nutrition, but also to inhibit endogenous synthesis of polyamines by using specific inhibitors of ornithine decarboxylase (ODC), of spermidine-spermine N1-acetyltransferase or of spermine oxidase, key enzymes in polyamine metabolism.

“Specific inhibitor” denotes a molecule capable of blocking, completely or partially, directly or indirectly, reversibly or irreversibly, the active site of at least one of the enzymes involved in the synthesis of polyamines in the human body, or animal body. The role of the inhibitor of biosynthesis of the polyamines is to stop or significantly reduce the endogenous production of polyamines in the body treated with the product according to the present invention. The combined use of an inhibitor of polyamine biosynthesis and of a food intake depleted of polyamines makes it possible to reduce the quantity of bioavailable polyamines in the body.

The compositions of the invention optionally contain an inhibitor of the intracellular synthesis of polyamines at a rate of at most 15 wt % relative to the total dry weight of the composition.

According to a more particular embodiment, the inhibitor of the intracellular synthesis of polyamines of the compositions of the invention is an inhibitor of ornithine decarboxylase, of spermidine-spermine N1-acetyltransferase or of spermine oxidase.

Among the inhibitors of ODC, alpha-difluoromethylornithine (α-DFMO) constitutes a compound that can be used, which is well known to a person skilled in the art (Fabian et al., 2002, Clin Cancer Res, 8(10), 3105-3117/Levin et al., 2003, Clin. Cancer Res., 9(3), 981-990/Meyskens et al., 2008, Cancer Prey. Res., 1(1), 32-38). This example must in no case limit the choice of an inhibitor of endogenous synthesis of the polyamines to this compound alone.

Other compounds able to inhibit ornithine decarboxylase, spermidine-spermine N1-acetyltransferase or spermine oxidase can be used. The quantities of inhibitors will be adapted by a person skilled in the art on the basis of the data on biological activity of these compounds and his general knowledge.

The transport of polyamines between the cell and the extracellular medium also allows fine regulation of the intracellular polyamines content. The combined use of an inhibitor of polyamine transport and of a food intake depleted of polyamines makes it possible to reduce the quantity of bioavailable polyamines in the body.

The compositions of the present invention are optionally enriched with at least one inhibitor of polyamine transport, at a rate of at most 15 wt % relative to the total dry weight of the composition.

According to a particular embodiment, the compositions of the present invention contain at least one inhibitor of the intracellular synthesis of polyamines, in particular selected from the inhibitors of ornithine decarboxylase, of spermidine-spermine N1-acetyltransferase or of spermine oxidase, and/or at least one inhibitor of polyamine transport, at a rate of at most 15 wt % relative to the total dry weight of the composition.

With the aim of further decreasing the endogenous synthesis of polyamines, it may be envisaged to have recourse to antibiotics in order to limit the intake of polyamines by the bacteria of the intestinal flora.

According to a particular embodiment, the compositions of the invention optionally contain at least one antibiotic.

This antibiotic may belong to the group of intestinal antiseptics, such as Ercefuryl®. In addition to its bacteriostatic or bactericidal effect, this antibiotic may also have an antiparasitic effect, such as Flagyl®.

The use of antibiotics may lead to a decrease in the intake of certain vitamins, in particular those supplied by the patient's intestinal flora. In this case, it may prove necessary to supplement the composition with these vitamins so as not to cause vitamin deficiencies in the patient in the case of prolonged administration of the composition.

“Deficiencies” denotes a lack of nutrients that may affect the physical or mental condition of a human or of an animal.

According to a particular embodiment, the compositions of the invention may be enriched with vitamins.

According to a more particular embodiment, the compositions of the present invention contain at least one antibiotic, and/or are enriched with vitamins.

Advantageously, the carbohydrates of the compositions of the invention belong to the group comprising glucose polymers, maltodextrins, sucrose, modified starches, glucose monohydrate, dehydrated glucose syrup, glycerol monostearate and mixtures thereof.

Advantageously, the proteins of the compositions of the invention belong to the group comprising soluble milk proteins, soya proteins, whey peptides, powdered egg white, potassium caseinate, non-phosphorylated peptides, casein peptides, mixed caseinate, soya isolate and mixtures thereof.

Advantageously, the fats of the compositions of the invention belong to the group comprising butter oil, peanut oil, medium-chain triglycerides, grapeseed oil, soya oil, evening primrose oil and mixtures thereof.

According to a particular embodiment, the fats of the compositions of the invention consist of a mixture of at least one oil of animal origin, at least one oil of vegetable origin and glycerol stearate.

According to a more particular embodiment, in the compositions of the present invention the carbohydrates belong to the group comprising glucose polymers, maltodextrins, sucrose, modified starches, glucose monohydrate, dehydrated glucose syrup, glycerol monostearate and mixtures thereof, and/or in which the proteins belong to the group comprising soluble milk proteins, soya proteins, whey peptides, powdered egg white, potassium caseinate, non-phosphorylated peptides, casein peptides, mixed caseinate, soya isolate and mixtures thereof, and/or in which the fats belong to the group comprising butter oil, peanut oil, medium-chain triglycerides, grapeseed oil, soya oil, evening primrose oil and mixtures thereof and/or in which the fats consist of a mixture of at least one oil of animal origin, at least one oil of vegetable origin and glycerol stearate.

In order to control the intake of exogenous source of polyamines and of agmatine, the compositions used according to the present invention must be able to constitute all or some of the patient's nutrition. In this sense they must provide an energy supply capable of meeting the patient's nutritional requirements.

When the patient is a human being, the compositions of the invention constitute the daily food ration of a human being and comprise:

from 75 g to 500 g of carbohydrates,

from 20 g to 185 g of fats,

from 20 g to 225 g of proteins,

vitamins, minerals and electrolytes in quantities sufficient to meet the daily nutritional requirements of a human being,

and optionally an inhibitor of the intracellular synthesis of polyamines at a rate of less than 50 g and preferably at a rate of from 1 to 10 g per day.

According to a particular embodiment, the compositions of the invention are a sub-multiple of a daily food ration of a human being and comprise:

from 75/X g to 500/X g of carbohydrates,

from 20/X g to 185/X g of fats,

from 20/X g to 225/X g of proteins,

vitamins, minerals and electrolytes in quantities sufficient to partially meet the daily nutritional requirements of a human being,

and optionally an inhibitor of the intracellular synthesis of polyamines at a rate of less than 50/X g and preferably at a rate of from 1/X to 10/X g per day,

where X is an integer between 2 and 8 and corresponds to the number of rations that have to be ingested by the patient to meet his daily nutritional requirements.

When the patient is an animal, the daily food ration is adapted as a function of the category and weight of the animal, whether it is a pet animal or a farm animal. The distribution of carbohydrates, fats and proteins as well as the requirements for vitamins, minerals and electrolytes in the daily food ration of an animal are well known to a person skilled in the art. Regarding the inhibitor of the intracellular synthesis of polyamines, the dose is adapted as a function of the animal's weight and optionally on the basis of data obtained in humans.

The compositions used according to the present invention may constitute the daily food ration or a sub-multiple of a daily food ration of an animal and will have to satisfy the daily nutritional requirements of an animal.

As an example, when the patient is a mouse, the compositions used according to the present invention constitute the daily food ration of a mouse and comprise:

from 0.6 g to 1.8 g of carbohydrates,

from 0.04 g to 1.2 g of fats,

from 0.01 g to 0.6 g of proteins,

vitamins, minerals and electrolytes in quantities sufficient to meet the daily nutritional requirements of an animal,

and optionally an inhibitor of the intracellular synthesis of polyamines at a rate of less than 300 mg and preferably at a rate of from 40 to 200 mg per day.

Just as for a human being, the compositions used according to the present invention may be a sub-multiple of a daily food ration of a mouse and comprise:
from 0.6/X g to 1.8/X g of carbohydrates,

from 0.04/X g to 1.2/X g of fats,

from 0.01/X g to 0.6/X g of proteins,

vitamins, minerals and electrolytes in quantities sufficient to partially meet the daily nutritional requirements of an animal,

and optionally an inhibitor of the intracellular synthesis of polyamines at a rate of less than 300/X mg and preferably at a rate of from 40/X to 200/X mg per day,

where X is an integer between 2 and 8 and corresponds to the number of rations that have to be ingested by the patient to meet its daily nutritional requirements.
The proportions of the constituents of the compositions of the present invention, given in the context of the nutrition of a mouse, are given as a guide and may serve as a basis for a person skilled in the art, who can adapt them, based on his general knowledge, to other animals.

The compositions of the invention may be in solid, liquid or semiliquid form. Their formulation is adapted as a function of the patient's capacity for absorbing nutrition by the oral route but also by the enteral or parenteral route. Thus, the compositions of the invention may be in the form of:
drinks, creams, precooked dishes, entremets or biscuits for use by the oral route;
lyophilized or spray-dried or dehydrated powder for reconstitution with water, of products in the form of drinks, creams, precooked dishes, entremets or biscuits for use by the oral route;
lyophilized or spray-dried or dehydrated powder for enrichment of conventional foodstuffs;
liquid packaged in sterile bags for use by the enteral route;
liquid and by compounds (fat, protein, carbohydrate and vitamins and minerals) in separate sterile bags for use by the parenteral route.

According to a particular embodiment, the compositions of the invention are in dry form, to be dissolved extemporaneously in a neutral vehicle.

According to a more particular embodiment, the compositions of the invention include a neutral vehicle, making them ready to use.

“Neutral vehicle” denotes an aqueous solution for making a more or less liquid composition, facilitating ingestion of the latter by the patient. Thus, the compositions of the invention will have a viscosity range extending from that of water to that of milk drinks for a temperature of from 4° C. to 40° C., at normal atmospheric pressure.

The compositions of the invention may be used directly as a medicament.

“Medicament” denotes any substance which, once ingested by the patient, provides him with a benefit in terms of wellbeing, improvement of biological markers, regression or remission of the pathology. By medicament is meant any substance or composition presented as having therapeutic or preventive properties in respect of human or animal diseases, as well as any product that may be administered to a human being or to an animal, with a view to establishing a medical diagnosis or restoring, correcting or modifying their organic functions (Article L5111-1 of the French Code of Public Health). A medicament generally consists of at least one active ingredient, i.e. a molecule endowing it with its therapeutic effects, and excipients.

The compositions of the invention may also be included in the formulation of pharmaceutical preparations.

The compositions of the invention may be used in the preparation of a medicament.

The compositions of the invention may be used as a food supplement or substitute.

The compositions of the invention may be used as a nutraceutical supplement.

Polyamine metabolism plays a key role in cellular proliferation. Knowing that a disturbance in the physiology of cell division is almost always harmful, it seems relevant to analyse the effects of a disturbance of polyamine metabolism in the context of pathologies involving processes of cellular hyperproliferation, and in particular in the context of cancer treatment.

The compositions of the invention may be used in the treatment, in a patient, of pathologies associated with cellular hyperproliferation.

The compositions of the invention may be used in the treatment, in a patient, of cancer.

According to a particular embodiment, the compositions of the invention may be used in the treatment, in a patient, of pathologies associated with cellular hyperproliferation, or in the treatment of cancer.

The compositions of the invention may be used in the treatment of pathologies associated with cellular hyperproliferation or in the treatment of cancer, said treatment being carried out by administering a unit dose ranging from 3.7 mg to 370 mg of agmatine.

“Unit dose” denotes the specific quantity of agmatine ingested by the patient in one dose. The unit dose is the appropriate presentation of a defined quantity of agmatine in a single-dose container, intended to be administered to the patient.

For maximum control of the exogenous intake of polyamines and agmatine, the compositions of the invention may serve as a basis for a diet that may comprise several phases, during which the exogenous intake of polyamines and agmatine is:
supplied completely by the compositions used according to the invention,
supplied predominantly by the compositions used according to the invention,
supplied partially by the compositions used according to the invention.

“Completely” denotes that the patient's nutrition is restricted to the compositions of the invention. No foodstuff other than the compositions of the invention is included in the patient's diet. During this phase, there is maximum control of the intake of polyamines and agmatine.

“Predominantly” denotes the possibility of introducing in the patient's diet a breakfast comprising foodstuffs with reduced polyamines content. The rest of the daily food ration is supplied by the compositions of the invention.

“Partially” denotes the possibility of introducing in the patient's diet a breakfast and at least one solid meal comprising foodstuffs with reduced polyamines content. The rest of the daily food ration is supplied by the compositions of the invention.

According to a particular embodiment, the compositions used according to the present invention are administered to the patient according to the following scheme:

(i) administration of a first dose of the composition of the invention for a first period of time and, consecutively,
(ii) administration of a second dose of the composition of the invention for a second period of time, the second dose being adjusted as a function of the patient's reaction to the first dose and, consecutively,
(iii) administration of a third dose of the composition of the invention for a third period of time, the third dose being adjusted as a function of the patient's reaction to the second dose.

“The patient's reaction” denotes his physiological capacity for deriving benefit from a diet depleted of polyamines and enriched with agmatine. The benefit may be assessed in particular from the regression of the tumoral mass. The improvement or disappearance of any sign of the disease may be evaluated by a clinical examination, biological tests or imaging carried out in the usual manner in the context of cancer. These criteria vary depending on the type of cancer. Three hypotheses are conceivable:
either the patient's physiological state improves after administration of a first dose of the composition, and in this case the second dose of the composition will contain at most the same levels of polyamines and agmatine as the first dose,
or the patient's physiological state shows no improvement after administration of a first dose of the composition, and in this case the second dose of the composition will contain at least the same levels of polyamines and agmatine as the first dose,
or the patient's physiological state has deteriorated after administration of a first dose of the composition, and in this case the second dose of the composition will contain levels of polyamines and agmatine either higher or lower than those of the first dose.
   The same kind of reasoning is applied for administration of the third dose. Thus, the practitioner has considerable latitude in the scheme for administration of the compositions of the invention.

According to a particular embodiment of the invention, the first period of time ranges from 7 to 14 days, in particular 7 days.

According to a particular embodiment of the invention, the second period of time ranges from 14 to 21 days, in particular 14 days.

According to a particular embodiment of the invention, the third period of time ranges from 28 to 63 days, in particular 63 days.

The first, second and third periods of time constitute a complete cycle of treatment of the patient. The compositions of the invention may be administered for one, two or more cycles. Repetition of these cycles is left to the practitioner's discretion.

According to another particular embodiment of the invention:
the first dose of agmatine ranges from 3.7 mg to 2.9 g of agmatine/d,
the second dose of agmatine ranges from 3.7 mg to 2.9 g of agmatine/d, and
the third dose of agmatine ranges from 3.7 mg to 2.9 g of agmatine/d.
   These doses correspond to the doses of agmatine supplied by 1 to 8 rations of a diet depleted of cadaverine, putrescine, spermidine and spermine and enriched with agmatine from a dose of 18.4 mg/kg of food to a dose of 1840 mg/kg of food.

The compositions of the present invention supply a daily quantity of agmatine from 3 times to 2230 times higher than that contained in an average food ration.

According to another particular embodiment of the invention:
the first dose of polyamines ranges from 9 ng to 146 ng of polyamines/d,
the second dose of polyamines ranges from 9 ng to 146 ng of polyamines/d, and
the third dose of polyamines ranges from 9 ng to 146 ng of polyamines/d.
   These doses correspond to the doses of agmatine supplied by 1 to 8 rations of a diet depleted of cadaverine, putrescine, spermidine and spermine and enriched with agmatine from a dose of 18.4 mg/kg of food to a dose of 1840 mg/kg of food.

The compositions of the present invention supply a daily quantity of polyamines from 260 times to 4200 times lower than that contained in an average food ration.

According to another particular embodiment of the invention:
the first dose of agmatine ranges from 3.7 mg to 2.9 g of agmatine/d and the first dose of polyamines ranges from 9 μg to 146 μg of polyamines/d,
the second dose of agmatine ranges from 3.7 mg to 2.9 g of agmatine/d and the second dose of polyamines ranges from 9 μg to 146 μg of polyamines/d, and
the third dose of agmatine ranges from 3.7 mg to 2.9 g of agmatine/d and the third dose of polyamines ranges from 9 μg to 146 μg of polyamines/d.
   These doses correspond to the doses of agmatine supplied by 1 to 8 rations of a diet depleted of cadaverine, putrescine, spermidine and spermine and enriched with agmatine from a dose of 18.4 mg/kg of food to a dose of 1840 mg/kg of food.

According to a particular embodiment, the present invention also relates to compositions comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
for use in the prevention or treatment, in a patient, of pathologies associated with cellular hyperproliferation, the total quantity of agmatine ingested per day by the patient not exceeding 11400 nanomoles per kcal of composition ingested, in particular 5000 nanomoles per kcal of composition ingested, in particular 1000 nanomoles per kcal of composition ingested, in particular 80 nanomoles per kcal of composition ingested.

According to a particular embodiment, the present invention also relates to compositions comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
   for use in the prevention or treatment, in a patient, of pathologies associated with cellular hyperproliferation, the total quantity of biologically active polyamines ingested per day by the patient not exceeding 0.42 nanomoles per kcal of composition ingested, in particular 0.30 nanomoles per kcal of composition ingested, in particular 0.25 nanomoles per kcal of composition ingested, in particular 0.21 nanomoles per kcal of composition ingested.

According to a particular embodiment, the present invention also relates to compositions comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
   for use in the prevention or treatment, in a patient, of pathologies associated with cellular hyperproliferation, the total quantity of agmatine ingested per day by the patient not exceeding 11400 nanomoles per kcal of composition ingested, in particular 5000 nanomoles per kcal of composition ingested, in particular 1000 nanomoles per kcal of composition ingested, in particular 80 nanomoles per kcal of composition ingested and the total quantity of biologically active polyamines ingested per day by the patient not exceeding 0.42 nanomole per kcal of composition ingested, in particular 0.30 nanomole per kcal of composition ingested, in particular 0.25 nanomole per kcal of composition ingested, in particular 0.21 nanomole per kcal of composition ingested.

In order to potentiate the effects of a first anticancer treatment, the compositions of the invention may be used as a second therapeutic agent.

The compositions of the invention may be administered to the patient before and/or during and/or after the anticancer treatment.

According to a particular embodiment, the present invention also relates to a combination of a composition comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
   and of a chemotherapeutic agent for use that is simultaneous, separate or spread over time for treating pathologies associated with cellular hyperproliferation, the total quantity of agmatine ingested per day by the patient not exceeding 11400 nanomoles per kcal of composition ingested, in particular 5000 nanomoles per kcal of composition ingested, in particular 1000 nanomoles per kcal of composition ingested, in particular 80 nanomoles per kcal of composition ingested.

According to a particular embodiment, the present invention also relates to a combination of a composition comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
   and of a chemotherapeutic agent for use that is simultaneous, separate or spread over time for treating pathologies associated with cellular hyperproliferation, the total quantity of biologically active polyamines ingested per day by the patient not exceeding 0.42 nanomole per kcal of composition ingested, in particular 0.30 nanomole per kcal of composition ingested, in particular 0.25 nanomole per kcal of composition ingested, in particular 0.21 nanomole per kcal of composition ingested.

According to a particular embodiment, the present invention also relates to a combination of a composition comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
   and of a chemotherapeutic agent for use that is simultaneous, separate or spread over time for treating pathologies associated with cellular hyperproliferation, the total quantity of agmatine ingested per day by the patient not exceeding 11400 nanomoles per kcal of composition ingested, in particular 5000 nanomoles per kcal of composition ingested, in particular 1000 nanomoles per kcal of composition ingested, in particular 80 nanomoles per kcal of composition ingested and the total quantity of biologically active polyamines ingested per day by the patient not exceeding 0.42 nanomole per kcal of composition ingested, in particular 0.30 nanomole per kcal of composition ingested, in particular 0.25 nanomole per kcal of composition ingested, in particular 0.21 nanomole per kcal of composition ingested.

According to a more particular embodiment, the present invention also relates to a combination of a composition comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
   and of a chemotherapeutic agent for use in the prevention or treatment, in a patient, of pathologies associated with cellular hyperproliferation.

### DESCRIPTION OF THE FIGURES

FIGS. 1-A and 1-B: Evolution of tumour volume

These figures show the variations of tumour volumes (in cm³, shown on the ordinate) as a function of the number of days (shown on the abscissa) elapsed after grafting said tumour in male C57BL/6 mice. The mice are grafted with a solid tumour of cells from Lewis lung carcinoma.

FIG. 1-A: Four groups of five mice are fed different diets 6 days after grafting took place. The start of treatment is indicated by the arrow on the graph.

The curve plotted with black circles represents the mice fed a diet containing a normal level of polyamines (125 mg/kg of food or 861 nmol/g of food).

The curve plotted with open circles represents the mice fed a diet containing a normal level of polyamines (125 mg/kg of food or 861 nmol/g of food), and drinking water containing neomycin (2 mg/ml).

The curve plotted with black squares represents the mice fed a diet depleted of cadaverine, putrescine, spermidine and spermine (51 μg/kg of food or 0.350 nmol/g of food).

The curve plotted with empty squares represents the mice fed a diet depleted of cadaverine, putrescine, spermidine and spermine (51 μg/kg of food or 0.350 nmol/g of food), and drinking water containing neomycin (2 mg/ml).

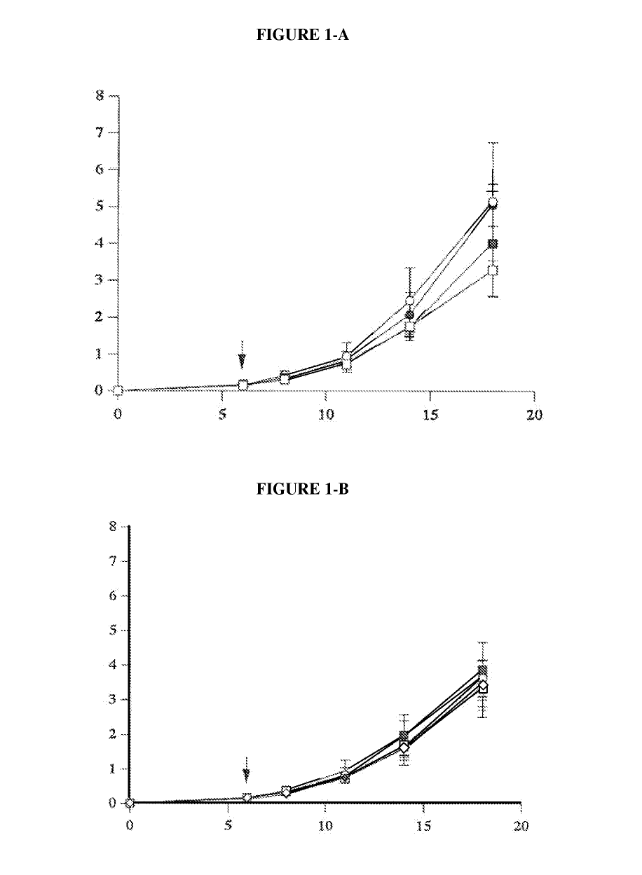

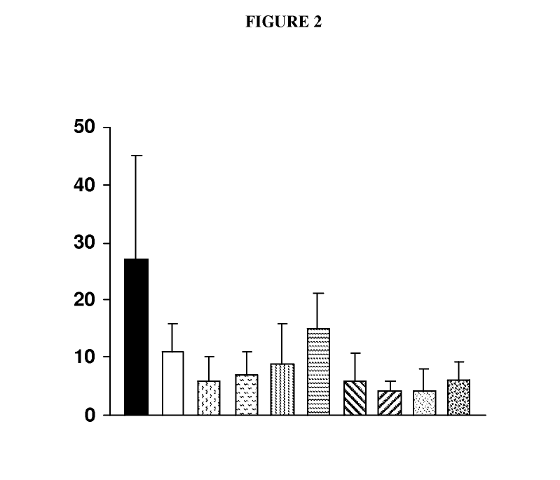

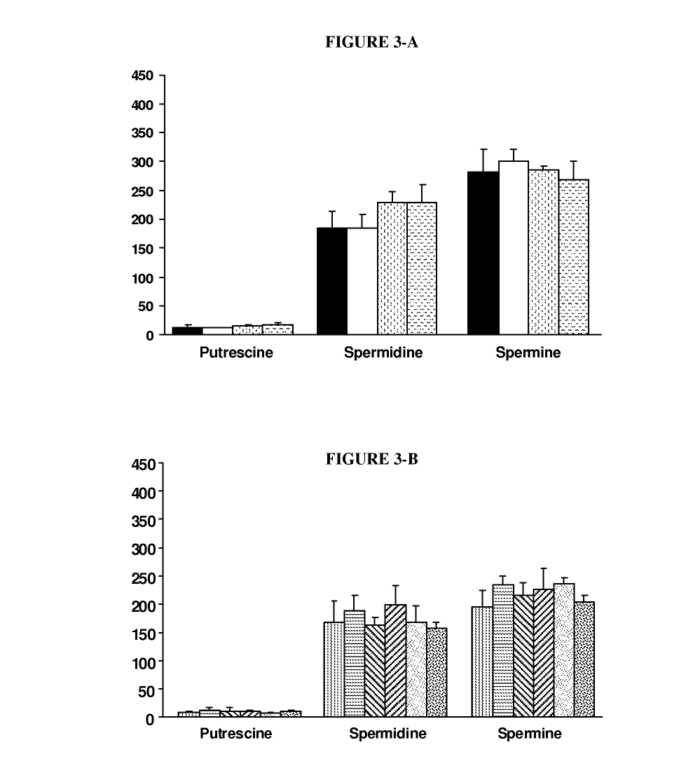

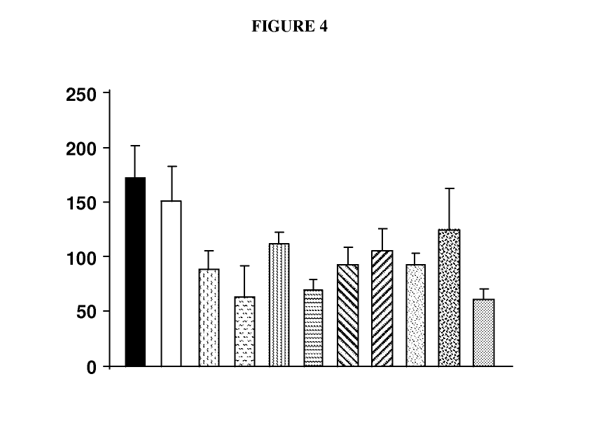

FIG. 1-B: Six groups of five mice are fed different diets 6 days after grafting took place. The start of treatment is indicated by the arrow on the graph.

The curve plotted with black circles represents the mice fed a diet depleted of cadaverine, putrescine, spermidine and spermine (51 μg/kg of food or 0.350 nmol/g of food), and enriched with agmatine (18.4 mg/kg of food or 141 nmol/g of food).

The curve plotted with open circles represents the mice fed a diet depleted of cadaverine, putrescine, spermidine and spermine (51 μg/kg of food or 0.350 nmol/g of food), enriched with agmatine (18.4 mg/kg of food or 141 nmol/g of food) and drinking water containing neomycin (2 mg/ml).

The curve plotted with black squares represents the mice fed a diet depleted of cadaverine, putrescine, spermidine and spermine (51 μg/kg of food or 0.350 nmol/g of food) and enriched with agmatine (184 mg/kg of food or 1410 nmol/g of food).

The curve plotted with empty squares represents the mice fed a diet depleted of cadaverine, putrescine, spermidine and spermine (51 μg/kg of food or 0.350 nmol/g of food), enriched with agmatine (184 mg/kg of food or 1410 nmol/g of food) and drinking water containing neomycin (2 mg/ml).

The curve plotted with black diamonds represents the mice fed a diet depleted of cadaverine, putrescine, spermidine and spermine (51 μg/kg of food or 0.350 nmol/g of food) and enriched with agmatine (1840 mg/kg of food or 14100 nmol/g of food).

The curve plotted with empty diamonds represents the mice fed a diet depleted of cadaverine, putrescine, spermidine and spermine (51 μg/kg of food or 0.350 nmol/g of food), enriched with agmatine (1840 mg/kg of food or 14100 nmol/g of food) and drinking water containing neomycin (2 mg/ml).

FIG. 2: Evolution of pulmonary metastatic dissemination

This figure shows the percentage pulmonary metastatic invasion (on the ordinate) in male C57BL/6 mice, as a function of the diet (on the abscissa) given to mice that had received grafts of a solid tumour of Lewis carcinoma cells. The percentage pulmonary metastatic invasion is measured 19 days after grafting took place.

Ten groups of mice are fed different diets.

The first group (represented by a black column) is fed a diet containing a normal level of polyamines (125 mg/kg of food or 861 nmol/g of food). The mean value of pulmonary metastatic invasion in the mice in this group is 27%.

The second group (represented by a white column) is fed a diet containing a normal level of polyamines (125 mg/kg of food or 861 nmol/g of food), and drinking water containing neomycin (2 mg/ml). The mean value of pulmonary metastatic invasion in the mice in this group is 11%.

The third group (represented by a column containing vertical broken lines) is fed a diet depleted of cadaverine, putrescine, spermidine and spermine (51 μg/kg of food or 0.350 nmol/g of food). The mean value of pulmonary metastatic invasion in the mice in this group is 6%.

The fourth group (represented by a column containing horizontal broken lines) is fed a diet depleted of cadaverine, putrescine, spermidine and spermine (51 μg/kg of food or 0.350 nmol/g of food), and drinking water containing neomycin (2 mg/ml). The mean value of pulmonary metastatic invasion in the mice in this group is 7%.

The fifth group (represented by a column containing vertical continuous lines) is fed a diet depleted of cadaverine, putrescine, spermidine and spermine (51 μg/kg of food or 0.350 nmol/g of food), and enriched with agmatine (18.4 mg/kg of food or 141 nmol/g of food). The mean value of pulmonary metastatic invasion in the mice in this group is 9%.

The sixth group (represented by a column containing horizontal continuous lines) is fed a diet depleted of cadaverine, putrescine, spermidine and spermine (51 μg/kg of food or 0.350 nmol/g of food), enriched with agmatine (18.4 mg/kg of food or 141 nmol/g of food) and drinking water containing neomycin (2 mg/ml). The mean value of pulmonary metastatic invasion in the mice in this group is 15%. The seventh group (represented by a column containing continuous lines with an upward slant to the left) is fed a diet depleted of cadaverine, putrescine, spermidine and spermine (51 μg/kg of food or 0.350 nmol/g of food), and enriched with agmatine (184 mg/kg of food or 1410 nmol/g of food). The mean value of pulmonary metastatic invasion in the mice in this group is 6%.

The eighth group (represented by a column containing continuous lines with an upward slant to the right) is fed a diet depleted of cadaverine, putrescine, spermidine and spermine (51 μg/kg of food or 0.350 nmol/g of food), enriched with agmatine (184 mg/kg of food or 1410 nmol/g of food) and drinking water containing neomycin (2 mg/ml). The mean value of pulmonary metastatic invasion in the mice in this group is 4%.

The ninth group (represented by a column containing small dots) is fed a diet depleted of cadaverine, putrescine, spermidine and spermine (51 μg/kg of food or 0.350 nmol/g of food), and enriched with agmatine (1840 mg/kg of food or 14100 nmol/g of food). The mean value of pulmonary metastatic invasion in the mice in this group is 4%.

The tenth group (represented by a column containing large dots) is fed a diet depleted of cadaverine, putrescine, spermidine and spermine (51 μg/kg of food or 0.350 nmol/g of food), enriched with agmatine (1840 mg/kg of food or 14100 nmol/g of food) and drinking water containing neomycin (2 mg/ml). The mean value of pulmonary metastatic invasion in the mice in this group is 6%.

FIGS. 3-A and 3-B: Evolution of the tumour levels of polyamines

These figures show the tumour levels of putrescine, spermidine and spermine (on the ordinate) of male C57BL/6 mice, as a function of the diet (on the abscissa) given to mice that had received grafts of a solid tumour of Lewis carcinoma cells. The tumour levels of putrescine, spermidine and spermine are measured 19 days after grafting took place.

FIG. 3-A: Four groups of five mice are fed different diets 6 days after grafting took place.

The black column represents the mice fed a diet containing a normal level of polyamines (125 mg/kg of food or 861 nmol/g of food).

The white column represents the mice fed a diet containing a normal level of polyamines (125 mg/kg of food or 861 nmol/g of food), and drinking water containing neomycin (2 mg/ml).

The column containing vertical broken lines represents the mice fed a diet depleted of cadaverine, putrescine, spermidine and spermine (51 μg/kg of food or 0.350 nmol/g of food).

The column containing horizontal broken lines represents the mice fed a diet depleted of cadaverine, putrescine, spermidine and spermine (51 g/kg of food or 0.350 nmol/g of food), and drinking water containing neomycin (2 mg/ml).

FIG. 3-B: Six groups of five mice are fed different diets 6 days after grafting took place.

The column containing vertical continuous lines represents the mice fed a diet depleted of cadaverine, putrescine, spermidine and spermine (51 μg/kg of food or 0.350 nmol/g of food), and enriched with agmatine (18.4 mg/kg of food or 141 nmol/g of food).

The column containing horizontal continuous lines represents the mice fed a diet depleted of cadaverine, putrescine, spermidine and spermine (51 μg/kg of food or 0.350 nmol/g of food), enriched with agmatine (18.4 mg/kg of food or 141 nmol/g of food) and drinking water containing neomycin (2 mg/ml).

The column containing continuous lines with an upward slant to the left represents the mice fed a diet depleted of cadaverine, putrescine, spermidine and spermine (51 μg/kg of food or 0.350 nmol/g of food) and enriched with agmatine (184 mg/kg of food or 1410 nmol/g of food).

The column containing continuous lines with an upward slant to the right represents the mice fed a diet depleted of cadaverine, putrescine, spermidine and spermine (51 μg/kg of food or 0.350 nmol/g of food), enriched with agmatine (184 mg/kg of food or 1410 nmol/g of food) and drinking water containing neomycin (2 mg/ml).

The column containing small dots represents the mice fed a diet depleted of cadaverine, putrescine, spermidine and spermine (51 μg/kg of food or 0.350 nmol/g of food) and enriched with agmatine (1840 mg/kg of food or 14100 nmol/g of food).

The column containing large dots represents the mice fed a diet depleted of cadaverine, putrescine, spermidine and spermine (51 μg/kg of food or 0.350 nmol/g of food), enriched with agmatine (1840 mg/kg of food or 14100 nmol/g of food) and drinking water containing neomycin (2 mg/ml).

FIG. 4: Evolution of the weight of the spleen

This figure shows the weight of the spleen in mg (on the ordinate) of male C57BL/6 mice, as a function of the diet (on the abscissa) given to the mice that had or had not received grafts of a solid tumour of Lewis carcinoma cells. The weight of the spleen is measured 19 days after grafting took place.

Ten groups of five mice that had received grafts of a solid tumour of Lewis carcinoma cells are fed different diets. An eleventh group of five mice that had not received grafts and were fed a diet containing a normal level of polyamines makes it possible to establish a reference mean value of the weight of the spleen in the mice that were used in this study.

The first group (represented by a black column) is fed a diet containing a normal level of polyamines (125 mg/kg of food or 861 nmol/g of food). The mean value of the weight of the spleen of the mice in this group is 171 mg.

The second group (represented by a white column) is fed a diet containing a normal level of polyamines (125 mg/kg of food or 861 nmol/g of food), and drinking water containing neomycin (2 mg/ml). The mean value of the weight of the spleen of the mice in this group is 151 mg.

The third group (represented by a column containing vertical broken lines) is fed a diet depleted of cadaverine, putrescine, spermidine and spermine (51 μg/kg of food or 0.350 nmol/g of food). The mean value of the weight of the spleen of the mice in this group is 88 mg.

The fourth group (represented by a column containing horizontal broken lines) is fed a diet depleted of cadaverine, putrescine, spermidine and spermine (51 μg/kg of food or 0.350 nmol/g of food), and drinking water containing neomycin (2 mg/ml). The mean value of the weight of the spleen of the mice in this group is 63 mg.

The fifth group (represented by a column containing vertical continuous lines) is fed a diet depleted of cadaverine, putrescine, spermidine and spermine (51 μg/kg of food or 0.350 nmol/g of food), and enriched with agmatine (18.4 mg/kg of food or 141 nmol/g of food). The mean value of the weight of the spleen of the mice in this group is 111 mg.

The sixth group (represented by a column containing horizontal continuous lines) is fed a diet depleted of cadaverine, putrescine, spermidine and spermine (51 μg/kg of food or 0.350 nmol/g of food), enriched with agmatine (18.4 mg/kg of food or 141 nmol/g of food) and drinking water containing neomycin (2 mg/ml). The mean value of the weight of the spleen of the mice in this group is 69 mg.

The seventh group (represented by a column containing continuous lines with an upward slant to the left) is fed a diet depleted of cadaverine, putrescine, spermidine and spermine (51 μg/kg of food or 0.350 nmol/g of food), and enriched with agmatine (184 mg/kg of food or 1410 nmol/g of food). The mean value of the weight of the spleen of the mice in this group is 92 mg.

The eighth group (represented by a column containing continuous lines with an upward slant to the right) is fed a diet depleted of cadaverine, putrescine, spermidine and spermine (51 μg/kg of food or 0.350 nmol/g of food), enriched with agmatine (184 mg/kg of food or 1410 nmol/g of food) and drinking water containing neomycin (2 mg/ml). The mean value of the weight of the spleen of the mice in this group is 105 mg.

The ninth group (represented by a column containing small dots) is fed a diet depleted of cadaverine, putrescine, spermidine and spermine (51 μg/kg of food or 0.350 nmol/g of food), and enriched with agmatine (1840 mg/kg of food or 14100 nmol/g of food). The mean value of the weight of the spleen of the mice in this group is 92 mg.

The tenth group (represented by a column containing large dots) is fed a diet depleted of cadaverine, putrescine, spermidine and spermine (51 μg/kg of food or 0.350 nmol/g of food), enriched with agmatine (1840 mg/kg of food or 14100 nmol/g of food) and drinking water containing neomycin (2 mg/ml). The mean value of the weight of the spleen of the mice in this group is 124 mg.

The eleventh group (represented by a grey column), corresponding to the mice that had not received grafts, is fed a diet containing a normal level of polyamines (125 mg/kg of food or 861 nmol/g of food). The mean value of the weight of the spleen of the mice in this group is 61 mg.

### EXPERIMENTAL SECTION

### Example 1

### Effect of the Diet Depleted of Cadaverine, Putrescine, Spermidine and Spermine, and Enriched with Agmatine

### 1—Animal Model

1.1—Mouse Strain

Male C57BL/6 mice, aged 9 weeks and weighing 20 g (Janvier breeding station, Le Genest St Isle, France), are acclimatized for 1 week before the start of the study. Handling of the animals is carried out in accordance with the ethical directives for experiments and according to the recommendations of the European Biomedical Research Association.

The animals are housed at a rate of 5 individuals per cage under standardized conditions (21±1° C.; 60% relative humidity, cycles of 12 h of light, 12 h of darkness), and have access to food and water ad libitum.

1.2—Tumour Model

Cells from Lewis lung carcinoma (3LL) were obtained from the ECACC (European Collection of Cell Cultures) and grafted by intramuscular injection in the hind paws of male C57BL/6 mice by the method described previously (Hergueux J. et al., Exp. Cell Biol., 1983, 51(4), 181-191) to produce tumour cells. After 20 days, the tumour cells are collected and dispersed in PBS, counted and diluted to reach 0.5×10⁶ cells/100 μL, before being grafted in the right hind paw of the mice. The grafted mice are distributed randomly in the cages in groups of 5 individuals.

The tumour becomes palpable some days after grafting took place. The animals are sacrificed 19 days after grafting the tumour.

### 2—Nutrition

The food depleted of cadaverine, putrescine, spermidine and spermine is called RPCPSdS (Regime Pauvre en [Diet depleted of] Cadaverine Putrescine Spermidine Spermine), and is in the form of solutes. This food depleted of polyamines contains less than 51 μg of polyamines/kg of food or 0.350 nmol/g of food.

The rodent food containing a normal level of polyamines (125 mg of polyamines per kilogram of food or 861 nmol per g of food) corresponds to standard commercial nutrition (UAR, Usine d'Alimentation Rationnelle).

The products are distributed to the animals according to their profile and their nutritional requirements. A mouse consumes about 2 grams of nutrition per day.

The treatment is set up 6 days after grafting, when the tumour is palpable, and is administered 7 days a week and comprises the intake ad libitum of the normal nutrition or the test solutes as well as drinking water.

### 3—Results

3.1—Tumour Growth

The animal model (C57BL/6 mouse) and the tumour model (Lewis carcinoma) are described above. The mice are grafted with 0.5×10⁶ tumour cells, by intramuscular injection.

Tumour growth is quantified 6, 8, 11, 14 and 18 days after grafting took place, by measuring the volume of the tumour, by the method described previously (Moulinoux et al., Int J. Cancer, 1984, 34(2), 277-281).

The mice are divided into ten groups and different diets are administered to each group.

The first group is fed a diet containing a normal level of polyamines (125 mg/kg of food or 861 nmol/g of food).

The second group is fed a diet containing a normal level of polyamines (125 mg/kg of food or 861 nmol/g of food) and drinking water containing neomycin (2 mg/ml).

The third group is fed a diet depleted of cadaverine, putrescine, spermidine and spermine (51 μg/kg of food or 0.350 nmol/g of food).

The fourth group is fed a diet depleted of cadaverine, putrescine, spermidine and spermine (51 μg/kg of food or 0.350 nmol/g of food), and supplemented with neomycin (2 mg/ml).

The fifth group is fed a diet depleted of cadaverine, putrescine, spermidine and spermine (51 μg/kg of food or 0.350 nmol/g of food), and enriched with agmatine (18.4 mg/kg of food or 141 nmol/g of food).

The sixth group is fed a diet depleted of cadaverine, putrescine, spermidine and spermine (51 μg/kg of food or 0.350 nmol/g of food), enriched with agmatine (18.4 mg/kg of food or 141 nmol/g of food) and drinking water containing neomycin (2 mg/ml).

The seventh group is fed a diet depleted of cadaverine, putrescine, spermidine and spermine (51 μg/kg of food or 0.350 nmol/g of food), and enriched with agmatine (184 mg/kg of food or 1410 nmol/g of food).

The eighth group is fed a diet depleted of cadaverine, putrescine, spermidine and spermine (51 μg/kg of food or 0.350 nmol/g of food), enriched with agmatine (184 mg/kg of food or 1410 nmol/g of food) and drinking water containing neomycin (2 mg/ml).

The ninth group is fed a diet depleted of cadaverine, putrescine, spermidine and spermine (51 μg/kg of food or 0.350 nmol/g of food), and enriched with agmatine (1840 mg/kg of food or 14100 nmol/g of food).

The tenth group is fed a diet depleted of cadaverine, putrescine, spermidine and spermine (51 μg/kg of food or 0.350 nmol/g of food), enriched with agmatine (1840 mg/kg of food or 14100 nmol/g of food) and drinking water containing neomycin (2 mg/ml).

Neomycin is diluted in the drinking water (0.2% w/v) regardless of whether the animals are fed with nutrition in solid form of the dry pellet type, or with nutrition in liquid or semiliquid form, in particular in the form of solutes.

The evolution of tumour volume (in cm³) is shown in FIG. 1-A for the animals in groups 1 to 4 and in FIG. 1-B for the animals in groups 5 to 10.

Neomycin has no effect on the evolution of tumour volume when it is administered in the drinking water on the basis of a diet containing a normal level of polyamines

The diet depleted of cadaverine, putrescine, spermidine and spermine causes a 20% decrease in tumour growth on D19. This decrease reaches 35% when neomycin is added to this diet depleted of cadaverine, putrescine, spermidine and spermine (FIG. 1-A).

Agmatine supplementation of the diet depleted of cadaverine, putrescine, spermidine and spermine increases the antitumour effect of this diet depleted of cadaverine, putrescine, spermidine and spermine by 8%, 3% and 13% respectively for agmatine doses of 18.4, 184 and 1840 mg/kg of food (FIG. 1-B).

3.2—Pulmonary Metastatic Invasion

Immediately after sacrificing the animals, the lungs are removed, stored in 10% formol and the pulmonary metastases are visualized using a binocular magnifier. The metastatic dissemination is expressed as percentage invasion relative to the total surface area of the lungs. The percentage metastatic invasion was measured in the mice of the ten groups described above, each having received different nutrition. The results are illustrated in FIG. 2.

Neomycin supplied as a supplement to a diet containing a normal level of polyamines causes a 60% decrease in metastatic invasion relative to that observed in the mice that had not received this antibiotic.

The diet depleted of cadaverine, putrescine, spermidine and spermine causes a 79% decrease in metastatic invasion relative to that observed in the mice that had received a diet containing a normal level of polyamines. Addition of neomycin to a diet depleted of cadaverine, putrescine, spermidine and spermine does not lead to any additional benefit.

Agmatine supplementation of the diet depleted of cadaverine, putrescine, spermidine and spermine does not appear to bring any additional benefit in terms of metastatic process.

3.3—Tumour Levels of Polyamines

The level of polyamines is measured in the tumour formed at the site of inoculation of the Lewis carcinoma cells. The tumour is washed and then the cells of which it is composed are dispersed using a homogenizer of the Polytron® type, in the presence of 0.2M perchloric acid. After incubation for at least 16 h at 4° C., the cells are centrifuged for 15 minutes at 3500 rpm at 4° C. The supernatant that contains the polyamines is removed and is used for determination of the polyamines. The polyamines are determined by the method described previously (Seiler N et al., 1996, Cancer Research, 56, 5624-5630). The three principal polyamines present in mammals (putrescine, spermidine and spermine) were determined for the ten groups of mice that had each received different nutrition. The results are illustrated in FIG. 3.

Neomycin causes a 17% decrease in the level of putrescine, a 7% increase in the level of spermine and has no effect on the level of spermidine based on a normal diet.

Conversely, neomycin causes a 17% increase in the level of putrescine, a 24% increase in the level of spermidine and a 5% decrease in the level of spermine based on a diet depleted of cadaverine, putrescine spermidine and spermine.

The diet depleted of cadaverine, putrescine, spermidine and spermine induces an increase of 17% and 24% of the tumour levels of putrescine and spermidine respectively. This diet has no effect on the tumour level of spermine.

Agmatine supplementation of the diet depleted of cadaverine, putrescine, spermidine and spermine induces decreases in the tumour levels of putrescine, spermidine and spermine

At a dose of 18.4 mg of agmatine/kg of food, the tumour levels of putrescine, spermidine and spermine decrease by 35%, 10% and 31% respectively relative to the tumour levels of these two polyamines in the mice that had received a diet containing a normal level of polyamines

At a dose of 184 mg of agmatine/kg of food, the tumour levels of putrescine, spermidine and spermine decrease by 14%, 12% and 24% respectively relative to the tumour levels of these two polyamines in the mice that had received a diet containing a normal level of polyamines

At a dose of 1840 mg of agmatine/kg of food, the tumour levels of putrescine, spermidine and spermine decrease by 50%, 10% and 16% respectively relative to the tumour levels of these two polyamines in the mice that had received a diet containing a normal level of polyamines

3.4—Weight of the Spleen

The animals are euthanized, then the spleen is removed from the mice and weighed using a precision balance.

In addition to the ten groups of mice that each received a different diet, as described above, an eleventh group of animals was included for this study. These were male C57BL/6 mice having the same basic physiological characteristics as the mice in the other ten groups but that had not received grafts of Lewis carcinoma cells. The mice in this eleventh group were fed the diet containing a normal level of polyamines (125 mg/kg of food or 861 nmol/g of food). The weight of the spleen of these healthy mice (61 mg) serves as reference for an animal with no sign of inflammation.

The mice that received a graft of Lewis carcinoma cells and that were given a diet containing a normal level of polyamines have a spleen weight three times greater (171 mg) than that of the healthy mice fed the same diet.

Neomycin causes a decrease in the weight of the spleen in the mice in groups 2, 4 and 6. Conversely, for the mice in groups 8 and 10 that received a diet depleted of cadaverine, putrescine, spermidine and spermine and enriched with agmatine at respective doses of 184 mg/kg of food and 1840 mg/kg of food, neomycin causes an increase in the weight of the spleen.

The diet depleted of cadaverine, putrescine, spermidine and spermine induces a decrease of the order of 50% of the weight of the spleen in these mice in comparison with the grafted mice that received a diet containing a normal level of polyamines.

Addition of agmatine to the diet depleted of cadaverine, putrescine, spermidine and spermine has no additional effect on the weight of the spleen, regardless of the dose of agmatine added.

### Example 2

### Effect of the Diet Depleted of Cadaverine, Putrescine, Spermidine and Spermine, and Enriched with Agmatine in Combination with an Anticancer Drug

### 1—Animal Model

1.1—Mouse Strain

Male C57BL/6 mice, aged 9 weeks and weighing 20 g (Janvier breeding station, Le Genest St Isle, France), are acclimatized for 1 week before the start of the study. Handling of the animals is carried out in accordance with the ethical directives for experiments and according to the recommendations of the European Biomedical Research Association.

The animals are housed at a rate of 5 individuals per cage under standardized conditions (21±1° C.; 60% relative humidity, cycles of 12 h of light, 12 h of darkness), and have access to food and water ad libitum.

1.2—Tumour Model

Cells from Lewis lung carcinoma (3LL) were obtained from the ECACC (European Collection of Cell Cultures) and grafted by intramuscular injection in the hind paws of male C57BL/6 mice by the method described previously (Hergueux J. et al., Exp. Cell Biol., 1983, 51(4), 181-191) to produce tumour cells. After 20 days, the tumour cells are collected and dispersed in PBS, counted and diluted to reach 0.5×10⁶ cells/100 μL, before being grafted in the right hind paw of the mice. The grafted mice are distributed randomly in the cages in groups of 5 individuals.

The tumour becomes palpable some days after the grafting took place. The animals are sacrificed 19 days after grafting the tumour cells.

### 2—Nutrition

The food depleted of cadaverine, putrescine, spermidine and spermine is called RPCPSdS (Regime Pauvre en [Diet depleted of] Cadaverine Putrescine Spermidine Spermine) and is in the form of solutes. This food depleted of polyamines contains less than 51 μg of polyamines/kg of food.

Rodent food containing a normal level of polyamines (125 mg of polyamines per kilogram of food or 861 nmol of polyamines per g of food) corresponds to standard commercial nutrition (UAR, Usine d'Alimentation Rationnelle).

The products are distributed to the animals according to their profile and their nutritional requirements. A mouse consumes about 2 grams of nutrition per day.

The treatment is set up 6 days after grafting, when the tumour is palpable, and is administered 7 days a week and comprises the intake ad libitum of the normal nutrition or of the test solutes as well as drinking water.

### 3—Results

3.1—Tumour Growth

The animal model (C57BL/6 mouse) and the tumour model (Lewis carcinoma) are described above. The mice are grafted with 0.5×10⁶ tumour cells, by intramuscular injection in one of the hind paws.

Tumour growth is quantified 6, 8, 11, 14 and 18 days after grafting took place, by measuring the volume of the tumour, by the method described previously (Moulinoux et al., Int J. Cancer, 1984, 34(2), 277-281).

The mice are divided into fifteen groups of five mice and different diets are administered to each group.

The first group is fed a diet containing a normal level of polyamines (125 mg/kg of food or 861 nmol/g of food).

The second group is fed a diet containing a normal level of polyamines (125 mg/kg of food or 861 nmol/g of food) and cyclophosphamide at a dose of 9 mg·kg⁻¹·week⁻¹.

The third group is fed a diet containing a normal level of polyamines (125 mg/kg of food or 861 nmol/g of food) and cyclophosphamide at a dose of 90 mg·kg⁻¹·week⁻¹.

The fourth group is fed a diet depleted of cadaverine, putrescine, spermidine and spermine (51 μg/kg of food or 0.350 nmol/g of food).

The fifth group is fed a diet depleted of cadaverine, putrescine, spermidine and spermine (51 μg/kg of food or 0.350 nmol/g of food) and cyclophosphamide at a dose of 9 mg·kg⁻¹·week⁻¹.

The sixth group is fed a diet depleted of cadaverine, putrescine, spermidine and spermine (51 μg/kg of food or 0.350 nmol/g of food) and cyclophosphamide at a dose of 90 mg·kg⁻¹·week⁻¹.

The seventh group is fed a diet depleted of cadaverine, putrescine, spermidine and spermine (51 μg/kg of food or 0.350 nmol/g of food), and enriched with agmatine (18.4 mg/kg of food or 141 nmol/g of food).

The eighth group is fed a diet depleted of cadaverine, putrescine, spermidine and spermine (51 μg/kg of food or 0.350 nmol/g of food), and enriched with agmatine (184 mg/kg of food or 1410 nmol/g of food).

The ninth group is fed a diet depleted of cadaverine, putrescine, spermidine and spermine (51 μg/kg of food or 0.350 nmol/g of food), and enriched with agmatine (1840 mg/kg of food or 14100 nmol/g of food).

The tenth group is fed a diet depleted of cadaverine, putrescine, spermidine and spermine (51 μg/kg of food or 0.350 nmol/g of food), enriched with agmatine (18.4 mg/kg of food or 141 nmol/g of food) and cyclophosphamide at a dose of 9 mg·kg⁻¹·week⁻¹.

The eleventh group is fed a diet depleted of cadaverine, putrescine, spermidine and spermine (51 μg/kg of food or 0.350 nmol/g of food), enriched with agmatine (184 mg/kg of food or 1410 nmol/g of food) and cyclophosphamide at a dose of 9 mg·kg⁻¹·week⁻¹.

The twelfth group is fed a diet depleted of cadaverine, putrescine, spermidine and spermine (51 μg/kg of food or 0.350 nmol/g of food), enriched with agmatine (1840 mg/kg of food or 14100 nmol/g of food) and cyclophosphamide at a dose of 9 mg·kg⁻¹·week⁻¹.

The thirteenth group is fed a diet depleted of cadaverine, putrescine, spermidine and spermine (51 μg/kg of food or 0.350 nmol/g of food), enriched with agmatine (18.4 mg/kg of food or 141 nmol/g of food) and cyclophosphamide at a dose of 90 mg·kg⁻¹·week⁻¹.

The fourteenth group is fed a diet depleted of cadaverine, putrescine, spermidine and spermine (51 μg/kg of food or 0.350 nmol/g of food), enriched with agmatine (184 mg/kg of food or 1410 nmol/g of food) and cyclophosphamide at a dose of 90 mg·kg⁻¹·week⁻¹.

The fifteenth group is fed a diet depleted of cadaverine, putrescine, spermidine and spermine (51 μg/kg of food or 0.350 nmol/g of food), enriched with agmatine (1840 mg/kg of food or 14100 nmol/g of food) and cyclophosphamide at a dose of 90 mg·kg⁻¹·week⁻¹.

The evolution of tumour volume (in cm³) is shown in Table 1 for the animals in groups 1 to 9.

TABLE 1
Evolution of tumour volume

| | | | | | | | | | | Tumour |
|---|---|---|---|---|---|---|---|---|---|---|
| | | | | | | | | | | volume on |
| | | | | | | | | | | D18 (% |
| | | Monitoring of tumour volume (in cm³) | | | | | | | | relative to |
| Group | Details of the group | D0 | D2 | D5 | D8 | D10 | D12 | D15 | D18 | UAR) |
| 1 | UAR | 0 | 0.1 | 0.25 | 0.37 | 0.8 | 1.5 | 3.3 | 3.7 | 0 |
| 2 | UAR + CY 9 mg · kg⁻¹ · week⁻¹ | 0 | 0.08 | 0.2 | 0.296 | 0.64 | 1.2 | 2.64 | 3.0 | −20% |
| 3 | UAR + CY 90 mg · kg⁻¹ · week⁻¹ | 0 | 0.02 | 0.05 | 0.074 | 0.16 | 0.3 | 0.66 | 0.7 | −81% |
| 4 | RPCPSdS | 0 | 0.065 | 0.1625 | 0.2405 | 0.52 | 0.975 | 2.145 | 2.4 | −35% |
| 5 | RPCPSdS + CY 9 mg · kg⁻¹ · week⁻¹ | 0 | 0.055 | 0.1375 | 0.2035 | 0.44 | 0.825 | 1.815 | 2.0 | −45% |
| 6 | RPCPSdS + CY 90 mg · kg⁻¹ · week⁻¹ | 0 | 0.01 | 0.025 | 0.037 | 0.08 | 0.15 | 0.33 | 0.4 | −90% |
| 7 | RPCPSdS + Ag 18.4 mg · kg⁻¹ | 0 | 0.057 | 0.143 | 0.211 | 0.456 | 0.855 | 1.881 | 2.1 | −43% |
| 8 | RPCPSdS + Ag 184 mg · kg⁻¹ | 0 | 0.062 | 0.155 | 0.229 | 0.496 | 0.930 | 2.046 | 2.3 | −38% |
| 9 | RPCPSdS + Ag 1840 mg · kg⁻¹ | 0 | 0.052 | 0.130 | 0.192 | 0.416 | 0.780 | 1.716 | 1.9 | −49% |

For groups 10, 11 and 12, an improvement of from 5 to 30% relative to group 5 is observed.

For groups 13, 14 and 15, an improvement of from 1 to 10% relative to group 6 is observed.

Cyclophosphamide (Endoxan®) administered at 90 mg·kg⁻¹·week⁻¹ has a considerable anti-proliferative effect regardless of the nutrition supplied. The percentage inhibition is of the order of 81 to 90%.

A low dose of cyclophosphamide (Endoxan®) of 9 mg·kg⁻¹·week⁻¹ combined with a diet depleted of cadaverine, putrescine, spermidine and spermine makes it possible to increase antitumour efficacy by 25% relative to a diet containing a normal level of polyamines (125 mg/kg of food or 861 nmol/g of food) combined with 9 mg·kg⁻¹·week⁻¹ of cyclophosphamide.

This diet depleted of cadaverine, putrescine, spermidine and spermine and enriched with agmatine makes possible a dose-dependent increase in the antitumour effect of cyclophosphamide at 9 mg·kg⁻¹·week⁻¹ of from 5 to 14%, depending on the doses of agmatine.

This same diet makes possible a dose-dependent increase in the antitumour effect of cyclophosphamide at 90 mg·kg⁻¹·week⁻¹ of from 1 to 10%, depending on the doses of agmatine.

These diets make it possible to potentiate the antitumour effect of low-dose cyclophosphamide, thus making it possible to limit its side-effects.

Antiproliferative effects with the compositions of the present invention can also be verified with other models of liquid or solid human tumours, in particular leukaemias by the HL60 line (Novak et al., Leukemia Research 35 (2011): 1248-1253), breast cancers by the MCF7 line (Wang et al. Biochemistry and Molecular Biology Reports 32 (1999): 173-180), prostate cancers by the DU145 line (Choi et al., Acta Pharmacologica Sinica 26 (2005): 616-622), bowel cancers by the lines Colo320, Colo205E, Cxl, SW480 (Molderings et al., Cancer 101 (2004): 858-68). The aforementioned cell lines are given as examples of certain types of cancer and must not restrict the scope of the present invention in any way.

1. Composition comprising a mixture of cadaverine, putrescine, spermine and spermidine at a concentration ranging from 0.31 to 0.63 nmol per gram of composition, said composition also comprising agmatine at a concentration ranging from 120 to 17100 nmol per gram of composition.

2. Composition according to claim 1, wherein the concentration of putrescine ranges from 0.19 to 0.32 nmol per gram of composition, and/or the concentration of cadaverine ranges from 0.02 to 0.08 nmol per gram of composition, and/or the concentration of spermine ranges from 0.009 to 0.03 nmol per gram of composition, and/or the concentration of spermidine ranges from 0.09 to 0.20 nmol per gram of composition.

3. Composition according to claim 1, which contains, in percentage dry weight relative to the total dry weight: 10% to 35% fats, 8% to 30% proteins, 35% to 80% carbohydrates, up to 10% of a mixture consisting of vitamins, minerals and electrolytes.

4. Composition according to claim 1, which contains at least one inhibitor of the intracellular synthesis of polyamines, in particular selected from the inhibitors of ornithine decarboxylase, of spermidine-spermine N1-acetyltransferase or of spermine oxidase, and/or at least one inhibitor of polyamine transport, at a rate of at most 15 wt % relative to the total dry weight of the composition.

5. Composition according to claim 1, which contains at least one antibiotic, and/or is enriched with vitamins.

6. Composition according to claim 3, in which the carbohydrates belong to the group comprising glucose polymers, maltodextrins, sucrose, modified starches, glucose monohydrate, dehydrated glucose syrup, glycerol monostearate and mixtures thereof, and/or in which the proteins belong to the group comprising soluble milk proteins, soya proteins, whey peptides, powdered egg white, potassium caseinate, non-phosphorylated peptides, casein peptides, mixed caseinate, soya isolate and mixtures thereof, and/or in which the fats belong to the group comprising butter oil, peanut oil, medium-chain triglycerides, grapeseed oil, soya oil, evening primrose oil and mixtures thereof and/or in which the fats consist of a mixture of at least one oil of animal origin, at least one oil of vegetable origin and glycerol stearate.

7. Composition according to claim 1, said composition constituting the daily food ration of a human being and comprising:
from 75 g to 500 g of carbohydrates,
from 20 g to 185 g of fats,
from 20 g to 225 g of proteins,
vitamins, minerals and electrolytes in quantities sufficient to meet the daily nutritional requirements of a human being,
and optionally an inhibitor of the intracellular synthesis of polyamines, or,
said composition being a sub-multiple of a daily food ration of a human being and comprising:
from 75/X g to 500/X g of carbohydrates,
from 20/X g to 185/X g of fats,
from 20/X g to 225/X g of proteins,
vitamins, minerals and electrolytes in quantities sufficient to partially meet the daily nutritional requirements of a human being,
and optionally an inhibitor of the intracellular synthesis of polyamines,
where X is an integer between 2 and 8 and corresponds to the number of rations that have to be ingested by the patient to meet his daily nutritional requirements.

8. Composition according to claim 1, said composition being in dry form, to be dissolved extemporaneously in a neutral vehicle.

9. Composition according to claim 1 for use as a medicament.

10. Pharmaceutical preparation comprising a composition according to claim 1.

11. Composition according to claim 1 for use as a food supplement or substitute, or as a nutraceutical supplement.

12. Composition according to claim 1 for use in the treatment, in a patient, of pathologies associated with cellular hyperproliferation, or in the treatment of cancer.

13. Composition for use according to claim 12, wherein the treatment is carried out by administering a unit dose ranging from 3.7 mg to 370 mg of agmatine.

14. Composition for use according to claim 12, wherein the composition is administered to the patient according to the following scheme:
(i) administration of a first dose of the composition of the invention for a first period of time and, consecutively,
(ii) administration of a second dose of the composition of the invention for a second period of time, the second dose being adjusted as a function of the patient's reaction to the first dose and, consecutively,
(iii) administration of a third dose of the composition of the invention for a third period of time, the third dose being adjusted as a function of the patient's reaction to the second dose.

15. Composition for use according to claim 14, wherein:
the first period of time ranges from 7 to 14 days, in particular 7 days, and/or
the second period of time ranges from 14 to 21 days, in particular 14 days, and/or
the third period of time ranges from 28 to 63 days, in particular 63 days.

16. Composition for use according to claim 14, wherein:
the first dose of agmatine ranges from 3.7 mg to 2.9 g of agmatine/d,
the second dose of agmatine ranges from 3.7 mg to 2.9 g of agmatine/d, and
the third dose of agmatine ranges from 3.7 mg to 2.9 g of agmatine/d.

17. Composition comprising:
a mixture of cadaverine, putrescine, spermidine and spermine at a concentration ranging from 0.31 to 0.63 nmol per gram of composition,
agmatine at a concentration ranging from 120 to 17100 nmol per gram of composition,
for use in the prevention or treatment, in a patient, of pathologies associated with cellular hyperproliferation, the total quantity of agmatine ingested per day by the patient not exceeding 11400 nanomoles per kcal of composition ingested, in particular 5000 nanomoles per kcal of composition ingested, in particular 1000 nanomoles per kcal of composition ingested, in particular 80 nanomoles per kcal of composition ingested.

18. Combination of a composition according to claim 17 and of a chemotherapeutic agent for use in the prevention or treatment, in a patient, of pathologies associated with cellular hyperproliferation.
